(19) United States
(12) Patent Application Publication
(10) Pub. No.: US 2012/0116540 A1
(43) Pub. Date: May 10, 2012

(54) Prosthetic foot and method of making

(75) Inventors: Andrew H. Hansen, Apple Valley, MN (US); Kerice A. Tucker, Evanston, IL (US)

(73) Assignee: Northwestern University

(21) Appl. No.: 13/200,703

(22) Filed: Sep. 28, 2011

Related U.S. Application Data
(63) Continuation-in-part of application No. 12/462,050, filed on Jul. 28, 2009.
(60) Provisional application No. 61/137,746, filed on Jul. 31, 2008.
(60) Provisional application No. 61/404,467, filed on Oct. 4, 2010.

(51) Int. Cl. A61F 2/66; B26D 3/00; B29C 39/00; B24B 1/00; B29C 45/00; B23P 17/04; B23B 35/00

(52) U.S. Cl. 623/53; 29/557; 83/13; 408/1 R; 451/36; 29/527.1; 264/239; 264/328.1; 264/299

(57) ABSTRACT

A prosthetic foot is made by machining or molding a prosthetic foot profile to have a plurality of slots in and along the length of the forefoot region of the foot shape to achieve the appropriate roll-over shape during walking, and slotting the heel region of the machined foot shape to provide shock absorption in early stance phase of walking.

### RELATED APPLICATIONS

This application is a continuation-in-part of copending U.S. Ser. No. 12/462,050 filed Jul. 28, 2009, which claims benefits and priority of provisional application Ser. No. 61/137,746 filed Jul. 31, 2008. the disclosures of both of which are incorporated herein by reference. This application also claims benefits and priority of U.S. provisional application Ser. No. 61/404,467 filed Oct. 4, 2010, the disclosure of which is incorporated herein by reference.

### CONTRACTUAL ORIGIN OF THE INVENTION

This invention was made with government support under Grant No. H133EO80009 awarded by the Department of Education (NIDRR). The government has certain rights in the invention.

### FIELD OF THE INVENTION

The present invention relates to a prosthetic foot that is amenable to rapid, low cost production and to a method of making the prosthetic foot.

### BACKGROUND OF THE INVENTION

Applicants previously have discovered that during walking, the physiological foot-ankle complex provides a roll-over shape, or effective “wheel-like” rocker, that can be modeled as an arc of a circle's. The radius of this circle seems invariant to different walking speeds, to added trunk weight, or when walking with shoes of different heel heights. The radius, however, appears to vary with the height of the person. Based on these observations, applicants developed the so-called Shape&Roll foot (see Sam, M., Childress, D., Hansen, A., Meier, M., Lambla, S., Grahn, E., Rolock, J. (2004) The Shape&Roll Prosthetic Foot (Part I): Design and Development of Appropriate Technology for Low-Income Countries. Medicine Conflict and Survival, Vol. 20, No. 4, 294-306.); a prosthetic foot that provides the appropriate stature-matched roll-over shape of the physiological foot-ankle complex, as described by Hansen A H. Roll-over Characteristics of Human Walking With Implications for Artificial Limbs. PhD Dissertation, Northwestern University, Evanston, Ill. U.S.A., 2002.

### SUMMARY OF THE INVENTION

The present invention provides a prosthetic foot that is easier to fabricate at lower cost while providing a desired roll-over shape during walking, and that can be used with commercially available cosmetic foot shells, making it more amenable to rapid production in low-income countries as well as industrialized countries.

An illustrative embodiment of the invention involves cutting or otherwise machining a three dimensional profile of the prosthetic foot shape from a solid block of material, such as a plastic plate, and then cutting or otherwise machining a plurality of slots in and along the length of the forefoot region of the machined foot shape to permit achievement of the appropriate roll-over shape during walking and slotting the heel region of the machined foot shape to provide shock absorption in the early stance phase of walking. A bore is drilled, cut or otherwise machined in the connector region of the foot shape to receive a connector adapter for the leg prosthesis to be connected to the foot.

The depth of the slots in the forefoot region of the foot shape is controlled to provide a desired compliance (stiffness) of the flexible foot plate of the machined foot shape during walking. The forefoot slots have open tops and sides. One or more forefoot slots can receive a respective stop element to adjust a flat region of the sole plate of the foot for stability in standing.

The present invention provides in another illustrative embodiment a prosthetic foot comprised of a one-piece (monolithic) shaped foot profile having a connector region with a bore to receive a connector adapter for a leg prosthesis, a forefoot region in front of the connector region with the forefoot region having a plurality of machined or molded slots along its length to provide the appropriate roll-over shape of the combined flexible foot plate, foot cosmesis, and shoe during walking, and a heel region below the connector region and having a machined or molded slot for shock absorption. The foot can be made by machining a solid block of suitable material or by molding material, such as injection molding or casting fluid material into a mold having the shape of the foot profile.

Further advantages of the present invention will become more apparent from the following detailed description taken in conjunction with the following drawings.

### BRIEF DESCRIPTION OF THE DRAWINGS

FIG. 1 is a schematic perspective view of a plastic plate or block showing schematically outlines of a foot shapes to be cut or otherwise machined out of the plastic plate.

FIG. 2 is a top elevation of the foot shape machined out of the plastic plate.

FIG. 3 is a side elevation of the foot shape machined out of the plastic plate.

FIG. 4 is a side elevation of the foot shape machined out of the plastic plate showing the bore machined in the connector region.

FIGS. 5 and 6 are perspective views from different directions of the foot shape machined out of the plastic plate.

FIG. 7 is a top elevation of the foot shape machined out of the plastic plate useful for a Symes amputation.

FIG. 8 is a side elevation of the foot shape of FIG. 7 machined out of the plastic plate.

FIG. 9 is a side elevation of the foot shape of FIG. 7 machined out of the plastic plate showing the bore machined in the connector region.

FIGS. 10 and 11 are perspective views from different directions of the foot shape of FIG. 7 machined out of the plastic plate.

FIG. 12 is a side elevation of the foot shape having a connector region and heel region with greater height machined out of the plastic plate.

FIGS. 13 and 14 are perspective views from different directions of the foot shape of FIG. 12 machined out of the plastic plate.

### DETAILED DESCRIPTION OF THE INVENTION

The present invention involves a Shape&Roll type prosthetic foot that is easy to fabricate at low cost while providing a desired roll-over shape during walking, and that can be used with commercially available cosmetic foot shells, making it more amenable to rapid production in low-income countries as well as industrialized countries.

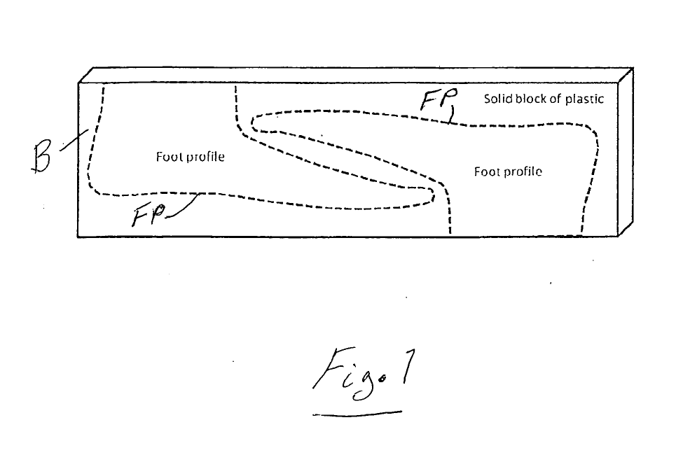

Pursuant to one embodiment of the present invention, the Shape&Roll prosthetic foot is made from a solid block B of plastic material as shown in FIG. 1. The solid block of plastic material can comprise polypropylene, polyethylene, HDPE, UHMWPE, VHMWPE, acetals, ABS, nylons, PVC, PEEK, Ultem, other polymers, suitable copolymers, or fiber-reinforced plastic materials and be in the form of a plastic plate having dimensions of 1.5 inches thickness, 4 inches width, and 4 feet length for purposes of illustration and not limitation. The thickness of the solid block B typically corresponds to the final thickness of the prosthetic foot, whereas the width and length are cut to form foot profile features.

In an illustrative embodiment of the invention, three dimensional foot profiles FP, FIG. 1, are be carved or cut out of the plastic block B using a simple X-Y operation on a CNC milling machine. Alternately, the foot profiles FP can be cut out of the solid block B by water jet machining device or laser machining device or any other suitable machining technique. When a CNC milling machine is used, the foot profiles FP that are cut from the solid block have sharp edges that are typicaly rounded. This edge rounding step can be conducted using quarter-round CNC tools.

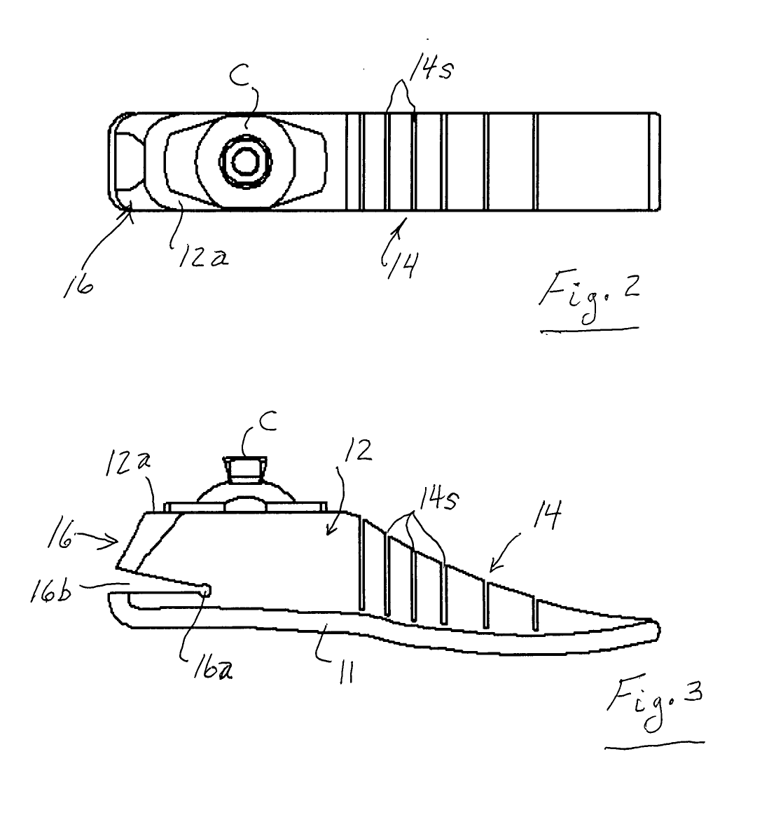

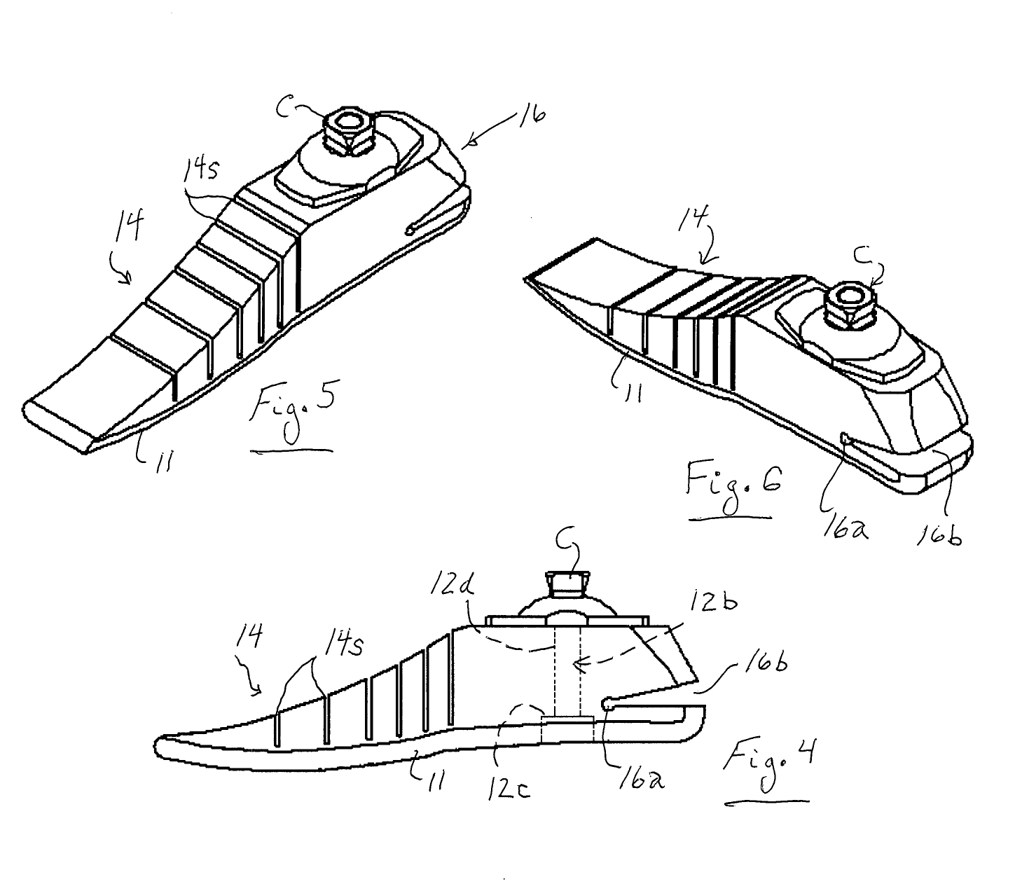

The foot profile shown in FIGS. 2-6 includes a machined connector block region 12 with a flat top surface 12a and with a forefoot region 14 fore (in front of) of the connector region 12 and a heel region 16 below and aft of the connector region. A foot sole plate 11 extends along the length of the prosthetic foot.

Referring to FIGS. 2-6, a plurality of slots 14s are cut or otherwise machined in and along the length of the forefoot region 14 of the machined foot profile FP to permit achievement of the appropriate roll-over shape during walking. The forefoot slots have open tops and sides as shown. The depth of the slots 14s in the forefoot region of the foot shape is controlled to provide a desired compliance (stiffness) of the flexible foot plate of the machined foot shape during walking. One or more forefoot slots 14s can receive a respective stop element (not shown) to adjust the length of the foot sole plate 11 for stability in standing as described in copending application Ser. No. 12/462,050, the teachings of which is incorporated herein by reference. The slots 14s typically are made by sawing using a band saw but can be by any suitable machining technique.

The heel region 16 of the machined foot profile is slotted as shown to provide shock absorption in the early stance phase of walking. In an illustrative embodiment of the invention, the heel region 16 is slotted by drilling or other machining a traverse hole 16a (perpendicular to the side plane of the foot profile) and removing a slot region 16b of the heel region adjacent and aft of the hole 16a.

A bore 12b, FIG. 4, is drilled, cut, or otherwise machined into the flat surface 12a of the connector block region 12 of the foot shape to receive a threaded bolt (not shown) that is threaded into the bottom of a connector adapter C for the leg prosthesis to be connected to the foot. For purposes of illustration and not limitation, the bore 12b includes a larger diameter lower bore 12c and a smaller diameter upper bore 12d to receive the threaded bolt for connection to a conventional pyramid adapter that resides on flat connector surface 12a to this end. The thickness of the block B can be chosen to interface with the pyramid connector adapter.

The final machined prosthetic foot is shown in FIGS. 2-6. The outer geometry of the foot profile FP is chosen to fit within a commercially available foot shell (not shown).

The present invention envisions performing other machining operations on the foot profile FP to reduce the weight of the device such as drilling additional holes through the side or carving out sections of plastic that are unnecessary and addition of indents on the side of the foot to indicate where the slots 14s should be machined as well as where the slots 14s should stop in depth for certain individual weight and activity levels.

The slots 14s can be made by the foot manufacturer or by a clinician fitting the foot to an individual pateint, but can also be cut deeper by the clinician if the patient finds the foot to be too stiff. Moreover, the transverse heel hole 16a can be increased in diameter by drilling with a larger drill bit, allowing the clinician to increase the diameter of the hole 16a to impart a softer heel (if the patient finds it is too stiff).

Applicants have found that certain thickest sole plate thickness can be used for different patients of different weights. These thickest sole plates 11 will just barely close the slots 14s in the appropriate sequence to create the desired appropriate rollover shape for that patient.

Moreover, the height of the foot profile can be adjusted by lowering the height of the connector block region for different foot shells and for different amputation levels, perhaps even to accommodate a Symes amputation. These changes require a corresponding change to the dimensions of the holes for the foot bolt.

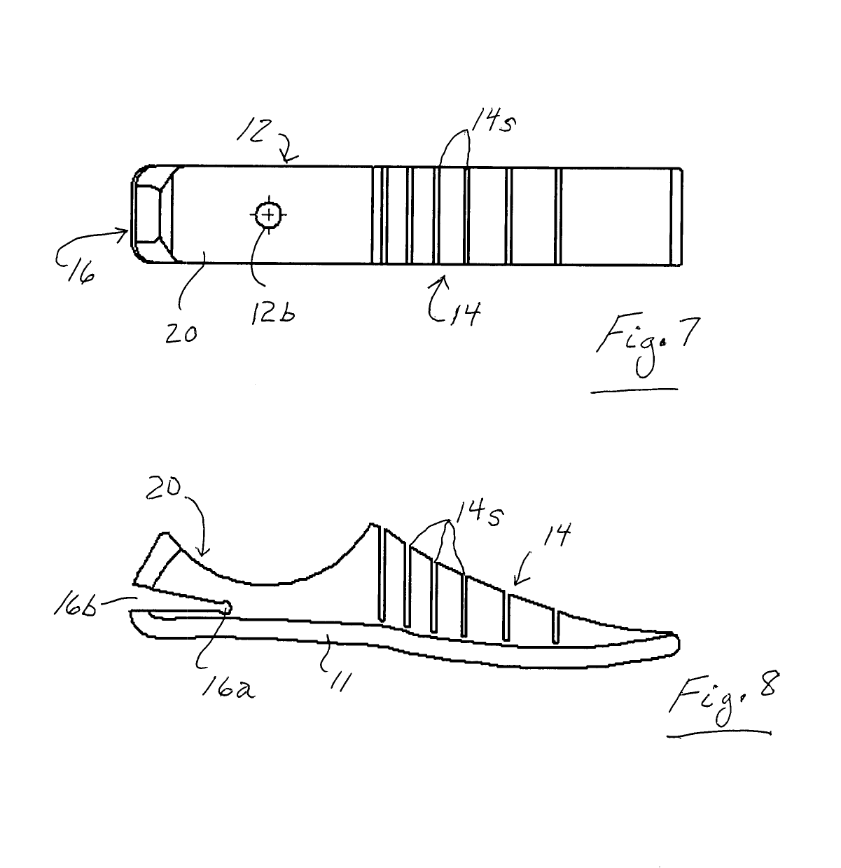

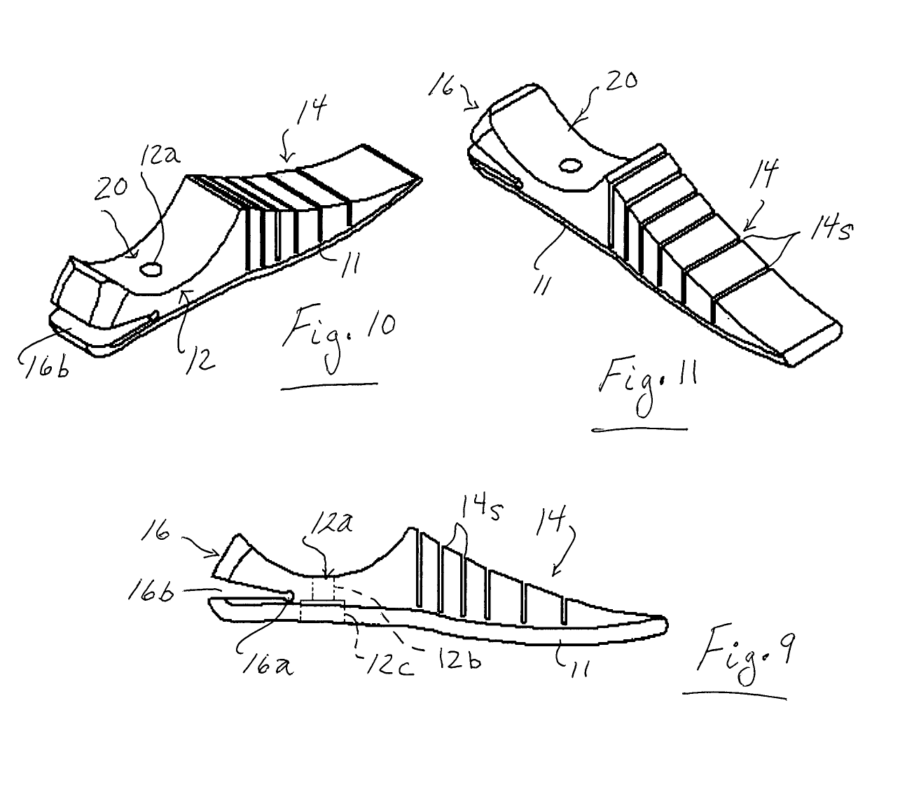

For example, FIGS. 7-11 illustrate a prosthetic foot pursuant to an embodiment of the invention modified for potential use with a Symes amputation. FIGS. 7-11 illustrate a prosthetic foot of an embodiment of the invention with a curved cutout 20 in the connector region 12 for receiving a socket (not shown) developed for a Symes amputee. Other features of the prosthetic foot of FIGS. 7-11 are similar to those of FIGS. 2-6 and are identified with similar reference numerals/characters. For example, the foot profile shown in FIGS. 7-11 is machined from block or plate of plastic material as described above. The machined foot profile includes a machined connector block region 12 with a flat top surface 12a and bores 12b and 12c, a forefoot region 14 with slots 14s fore (in front of) of the connector region 12, and a heel region 16 with transverse hole 16a and slot region 16b below and aft of the connector region. Attachment of the prosthetic foot to the Symes socket could be made alternately using a threaded bolt oriented in the opposite direction of the threaded bolt for FIGS. 2-6, or using gluing or bonding of the foot to the socket without a bolt. A foot sole plate 11 extends along the length of the prosthetic foot. The final machined prosthetic foot is shown in FIGS. 7-11.

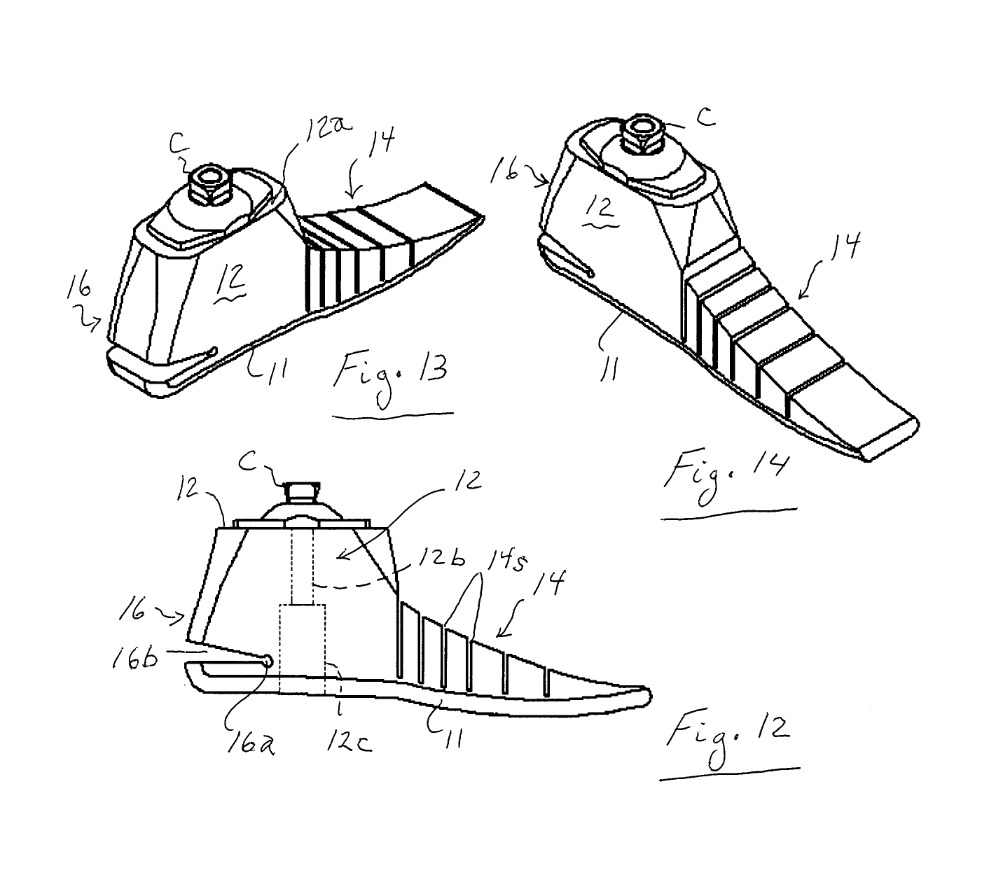

FIGS. 12-14 illustrate a foot profile having a connector region 12 and heel region 16 with greater height dimension machined out of the plastic plate for purposes of further illustration and not limitation. The foot profile shown in FIGS. 12-14 is machined from block or plate of plastic material as descried above. Other features of the prosethic foot of FIGS. 12-14 are similar to those of FIGS. 2-6 and are identified with similar reference numerals/characters. For example. the foot profile shown in FIGS. 12-14 includes a machined connector block region 12 with a greater height dimension and with a flat top surface 12a and bores 12b and 12c. a forefoot region 14 having slots 14s fore (in front of) of the connector region 12, and a heel region 16 with a greater height dimension and transverse hole 16a and slot region 16b below and aft of the connector region. The flat connector surface 12a receives thereon a connector adapter C for the leg prosthesis to be connected to the foot. A foot sole plate 11 extends along the length of the prosthetic foot.

The prosthetic foot pursuant to the invention is advantageous in that it can provide a low-cost alternative to many low-cost prosthetic feet that are sold around the world including feet such as the commercially available SACH foot, which have been shown to have inferior walking function compared to the Shape&Roll prosthetic foot. The prosthetic foot of the present invention can provide a more biomimetic walking function, a longer effective foot length, and reduced sound limb loading compared to the SACH feet. It is also expected that this foot will provide superior balance properties to the SACH foot when used together with the one or more stop elements received in one or more slots 14s as described in copending U.S. Ser. No. 12/462,050 filed Jul. 28, 2009, the disclosure of which is incorporated herein by reference.

Although the above description described machining of the three dimensional foot profile and features from a solid block of plastic or other suitable material, the present invention envisions molding material to the desired three dimensional foot profile, such as injection molding or casting fluid foot material into a mold having the shape corresponding to the foot profile. A combination of machining and molding can be used to produce the prosthetic foot. For example, the foot profile can be molded in simple manner and then the forefoot slots, heel hole/slot, and connector bores can be machined into the molded one-piece foot profile.

Although the present invention has been described in connection with certain embodiments of the invention, those skilled in the art will appreciate that modifications and changes can be made therein within the scope of the invention as set forth in the appended claims.

We claim:

1. A method of making a prosthetic foot, comprising machining a prosthetic foot profile from a solid block of material, machining a plurality of slots in and along the length of the forefoot region of the machined foot shape to achieve the appropriate roll-over shape during walking, and slotting the heel region of the machined foot shape to provide shock absorption during walking.

2. The method of claim 1 further including machining a bore in the connector region of the foot shape to receive a connector adapter for the leg prosthesis to be connected to the foot.

3. The method of claim 1 wherein the depth of the slots in the forefoot region of the foot shape is controlled to provide a desired stiffness of the flexible foot plate of the machined foot shape during walking.

4. The method of claim 1 wherein the forefoot slots have open tops and sides.

5. The method of claim 1 wherein the forefoot slots are machined by sawing.

6. The method of claim 1 wherein the heel region is slotted by drilling a traverse hole and removing a region of the heel region adjacent and aft of the hole.

7. The method of claim 1 wherein a outer geometry of the foot profile is machined to fit within a commercially available foot shell.

8. The method of claim 1 wherein the foot profile is machined by water jet.

9. A method of making a prosthetic foot, comprising molding a prosthetic foot profile from a plastic material to have a plurality of slots in and along the length of the forefoot region of the foot profile to achieve the appropriate roll-over shape during walking and to have a slotted heel region to provide shock absorption during walking.

10. The method of claim 9 wherein the foot is injection molded.

11. The method of claim 9 wherein the foot is cast in a mold.

12. A method of making a prosthetic foot. comprising molding a prosthetic foot profile from a plastic material, machining a plurality of slots in and along the length of the forefoot region of the molded foot profile to achieve the appropriate roll-over shape during walking and slotting a heel region of the molded foot profile to provide shock absorption during walking.

13. The method of claim 9 wherein the foot profile is injection molded.

14. The method of claim 9 wherein the foot profile is cast in a mold.

15. A prosthetic foot comprised of a one-piece shaped outer foot profile having a connector region with a bore to receive a connector adapter for a leg prosthesis, a forefoot region fore of the connector region with the forefoot region having a plurality of slots along its length to provide the appropriate roll-over shape of the flexible foot plate during walking, and a heel region below the connector region and having an aft slot for shock absorption.

16. The foot of claim 15 having a bore in the connector region of the foot shape to receive a connector adapter for the leg prosthesis to be connected to the foot.

17. The foot of claim 15 wherein the depth of the slots in the forefoot region of the foot shape is controlled to provide a desired compliance (stiffness) of the flexible foot plate of the machined foot shape during walking.

18. The foot of claim 15 wherein the forefoot slots have open tops and sides.

19. The foot of claim 15 wherein the forefoot slots are machined by a sawing.

20. The foot of claim 15 wherein the heel region slot includes a drilled hole and a machined slot region adjacent and aft of the drilled hole.

21. The foot of claim 15 made of a polymer material, copolymer material, or fiber-reinforced plastic material.

22. The foot of claim 15 having an outer geometry of the foot profile to fit within a commercially available foot shell.

23. The foot of claim 15 having a machined one-piece foot profile.

24. The foot of claim 15 having a molded one-piece foot profile.
